(19) United States
(12) Patent Application Publication
(10) Pub. No.: US 2005/0119537 A1
(43) Pub. Date: Jun. 2, 2005

(54) Method and apparatus for performing concurrent multiple measurements of relative hydration

(76) Inventors: Michael J. E. Campbell, Meredith, NH (US); Richard W. Sheppe JR., Post Mills, VT (US)

Correspondence Address: GEORGE A. HERBSTER, 40 BEACH STREET, SUITE 303, MANCHESTER, MA, 01944, US

(21) Appl. No.: 10/962,213

(22) Filed: Oct. 8, 2004

Related U.S. Application Data
(60) Provisional application No. 60/509,574, filed on Oct. 8, 2003.

(51) Int. Cl. A61B 5/00; G01R 27/04; G01R 27/32; A61B 10/00; G06F 17/00

(52) U.S. Cl. 600/306; 600/300; 128/920

(57) ABSTRACT

A method and apparatus for obtaining concurrent measurements of relative hydration from multiple probes associated with dermal phase meter units. A processor iteratively receives data in a loop from a plurality of units. The processor displays the current measurements on a composite screen.

### BACKGROUND OF THE INVENTION

This invention generally relates to measurements of relative hydration of the human skin or physical substrate materials and more specifically to making multiple concurrent measurements of relative hydration in such environments.

### DESCRIPTION OF RELATED ART

There is a continuing growing interest in measuring the relative hydration of a substrate, such as measurements on human skin. Such measurements can provide information about wound healing. There is also evidence that such measurements can predict certain diseases or the effectiveness of treatment.

U.S. Pat. No. 6,370,426 issued Apr. 9, 2002 for a method and apparatus for measuring relative hydration of a substrate discloses a probe and related equipment that provides such measurements. Such a probe is also known as a dermal phase meter, or DPM unit. This particular probe has an elongated probe housing. A sensor body mounted at one end of the probe housing has first and second concentric electrodes for contacting a substrate at the site for which a measurement of relative hydration is desired. An electrical impedance measurement circuit in the probe housing generates an impedance signal representing the impedance of the substrate between the first and second electrodes. This particular probe includes a temperature sensor, as an example of an environmental sensor, for generating a signal representing the temperature of the skin contacting the electrodes. A signal processor in the probe housing polls the impedance measurement circuit and the temperature sensor to generate processed impedance and temperature measurement signals. A connector at the other end of the probe housing enables communications between a data processing system and the signal processor. In this particular embodiment the data processing system issues commands to control the operation of the probe.

Over time these probes have been used in a wide variety of applications including the measurement of mean arterial pressure, neonatal skin maturation, burn treatment and others. In some applications diagnosis requires measurements at different locations on a patient. If a single device is used, the measurements are taken serially. While the test itself does not generally produce discomfort, a patient, particularly a burn patient, can experience discomfort merely from being positioned for an extended period of time in an uncomfortable position. Probes of this type have been used in clinical trials for ascertaining the efficacy of relative hydration motions as indicators or predictors of certain types of diseases or treatment. Clinical trials may involve many people. A single probe then requires each person to be measured separately. This increases the time required for handling multiple subjects.

What is needed is a method and apparatus that enables all the data to be gathered in a single data processing system to facilitate concurrent measurements on one or multiple subjects.

### SUMMARY

Therefore it is an object of this invention to provide a method and apparatus for obtaining concurrent measurements of relative hydration with a single system with multiple probes.

In accordance with one aspect of this invention, readings from a plurality of probes are obtained in a data processing system with a corresponding plurality of serial ports and a display. A concordance establishes the relationship between each probe and one serial port. Each port and corresponding probe is then accessed to input data in accordance with the established concordance. The data is then read from the probe and displayed on a composite display.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a view of a system for measuring relative hydration in accordance with this invention;

FIG. 2 is an enlarged version of a display shown in FIG. 1;

FIG. 3 is a block diagram that depicts the organization of the system shown in FIG. 1; and

FIG. 4 is a flow chart depicting the operation of a control shown in FIG. 3.

### DESCRIPTION OF ILLUSTRATIVE EMBODIMENTS

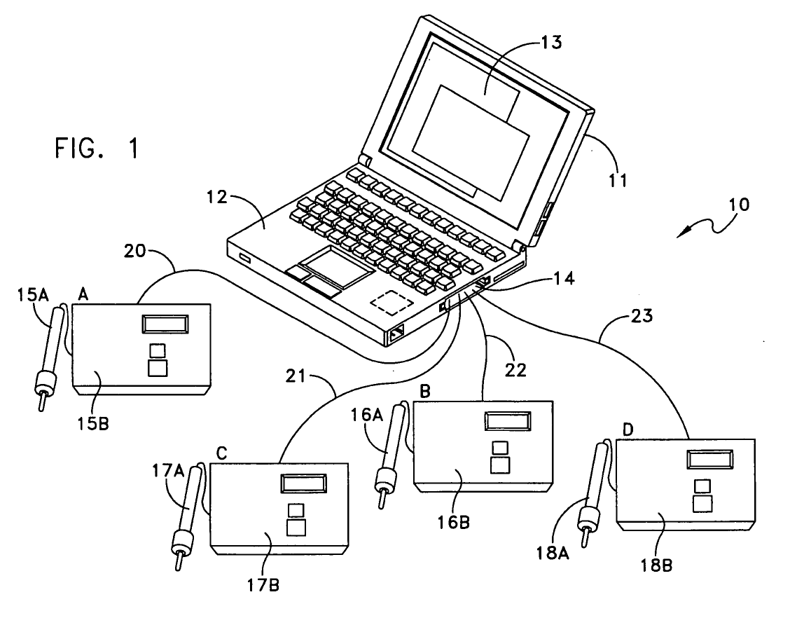

FIG. 1 depicts one embodiment 10 of a system constructed in accordance with this invention that includes a data processing system in the form of a laptop computer 11 with a keyboard 12 and a screen 13. The data processing system 11 includes a PCMCIA card 14 that can be a commercially available four-port asynchronous PCMCIA card that provides four inputs wherein each input can be treated as a serial port (e. g., a COM port).

FIG. 1 depicts four dermal phase meter (DPM) units 15 through 18 that comprise four probes 15A, 16A, 17A and 18A and corresponding interfaces 15B, 16B, 17B and 18B. Cables 20, 21, 22 and 23 connect each of the DPM units 15 through 18 to separate input channels of the PCMCIA card 14. FIG. 1 further defines the corresponding channels as channels A, B, C and D for the DPM units 15 through 18, respectively. The data processing system 11 produces a display on the screen 13 that dynamically presents measurement results.

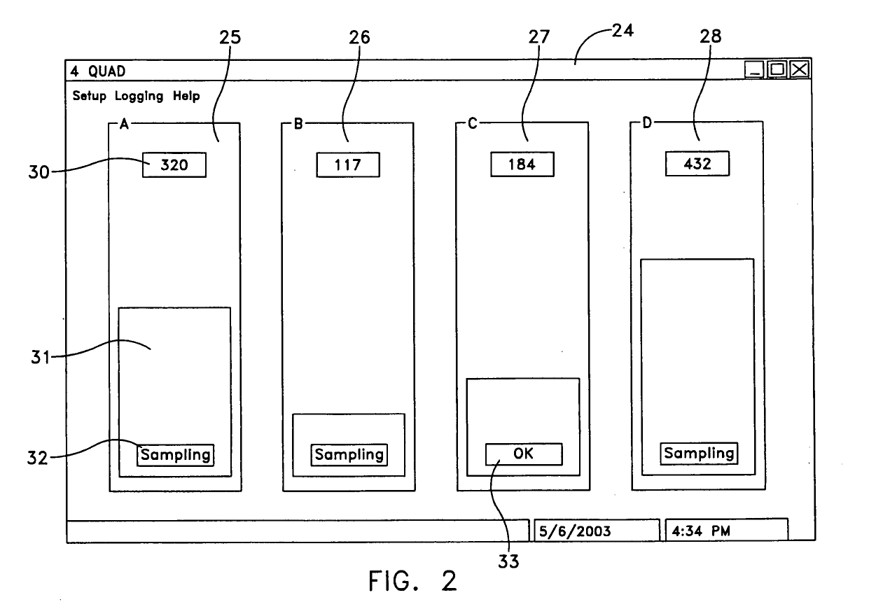

Referring to FIG. 2, the display 24 that appears on the screen 13 in FIG. 1 has one vertical area or column for each channel. That is, vertical areas 25, 26, 27 and 28 correspond to channels A through D, respectively that, in turn, may correspond to DPM units 15 through 18, respectively. As each vertical area is identical, only the vertical area for channel A, that is vertical area 25, is described in detail. This vertical channel 25 includes, as particularly shown in FIG. 2, a numerical display 30 that depicts a number. That number is the instantaneous value of measurement and is an indicator of the relative hydration of the skin being monitored by the corresponding DPM unit, such as the DPM unit 15. A graphical display 31 in a form of a dynamic bar graph display graphically depicts this level.

A status display 32 indicates whether sampling is continuing or whether the interval for which the test is being applied has been completed. In the particular display shown in FIG. 2, channels A, B and D are in the sampling mode while the sampling has been completed for channel C as depicted by the status display 33 in the vertical area 27 containing the message “OK”.

It will now be apparent that the information provided by the display 24 is a central display of four different measurements that can be taken concurrently. As previously indicated, these can represent the use of multiple DPM units in a single patient, or a single DPM unit on multiple patients or multiple DPM units on multiple patients.

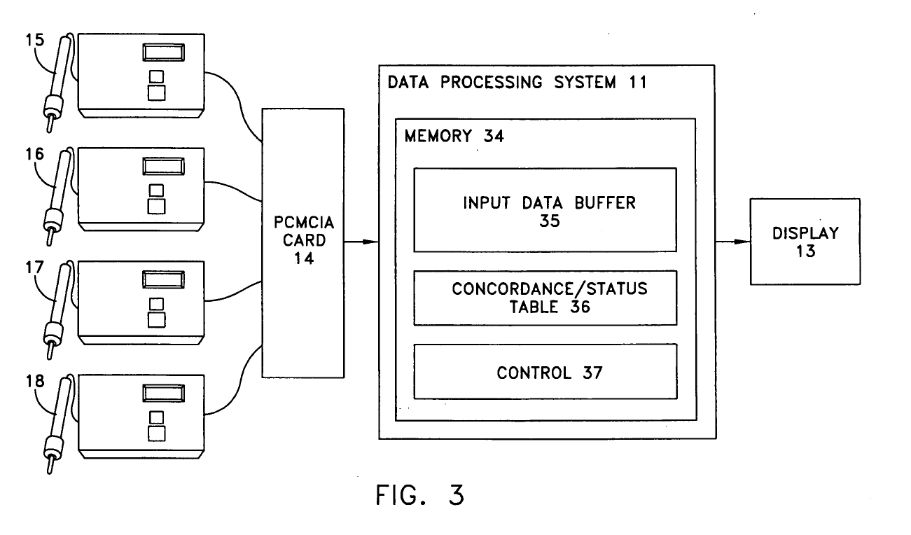

FIG. 3 depicts one implementation of the data processing system 11 that includes a memory 34 organized with an input data buffer 35, a concordance/status table 36 and a control 37. This is a functional organization of the data processing system. The actual implementation may be varied depending upon the various capabilities and processes being run by the data processing system 11. FIG. 3 depicts the DPM units 15 through 18 as inputs through the PCMCIA card 14 and the display 13 in the form of the screen 24 shown in FIGS. 1 and 2 as an output.

Still referring to FIG. 3, the input data buffer will receive data readings and any other selected information from the DPM units. In one embodiment the input data buffer can be partitioned into areas dedicated to each specific DPM unit and channel. In other embodiments the input data could be tagged with a channel marker or identifier thereby to allow the data to be stored in more random fashion or in an interlace fashion. Depending upon the particular application, the input data buffer could store all the readings. Alternately, the data could be stored in a separate file so that input data buffer 35 would be available for subsequent diagnoses.

The concordance/status table 36 establishes the correspondence or concordance between a probe, such as any one of the DPM units 15 through 18, and a channel represented by a COM port. This table allows any arbitrary positioning of the probes of each DPM unit with respect to channels. As previously indicated, there are multiple states attained during a measurement. The status of each measurement can also be maintained in the concordance status table 36 or as a separate table.

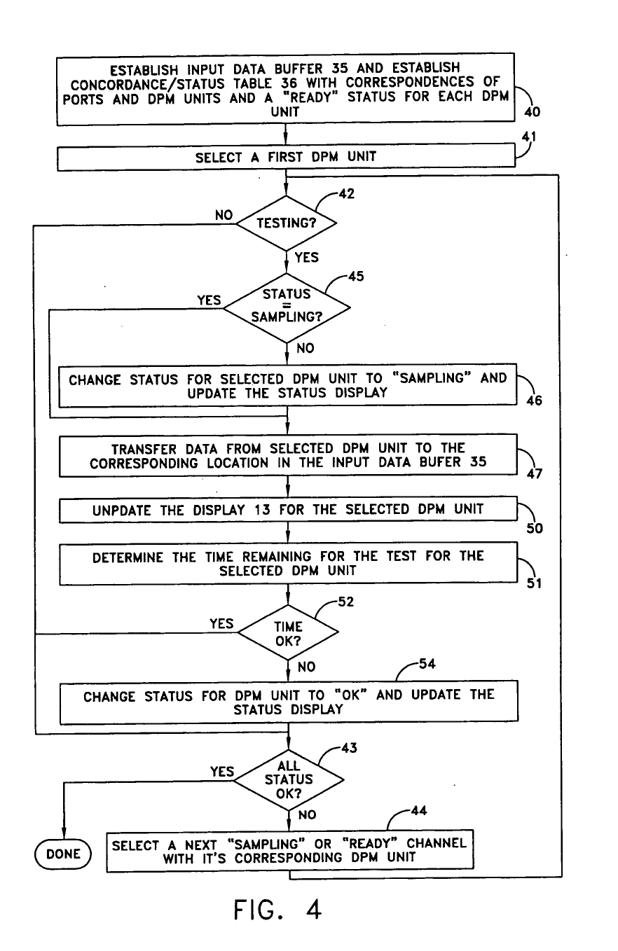

The control 37 manages the operation of the data processing system for the purposes of implementing this invention. FIG. 4 depicts one embodiment as a series of steps defining one functional implementation of the control 37 that has been found to be advantageous for implementing this invention. Referring to FIG. 4, steps 40 and 41 represent the processes for initializing a system. Step 40 specifically establishes the input data buffer as a work space within the memory 34 and establishes the concordance status table 36 including the correspondences between the ports and the DPM units. Step 40 also initializes the status for each DPM unit to an initial, or “READY”, state. Step 41 then selects a first probe or DPM unit.

Once the initialization is complete, the operation of FIG. 4 enters a loop so the probes are polled in an iteration function. Step 42 determines whether the probe is in fact performing a test, i.e., is in a “testing” mode. If testing is not underway, step 42 transfers control to step 43 that, during an initial iteration, will transfer control to step 44 to select a next channel and a corresponding DPM unit and then transfer control back to step 42. This effectively disables the remainder of the loop in FIG. 4. Step 43 performs this function because none of the DPM units will indicate an “OK” status at this initial iteration.

When a channel is in the testing mode, control transfers to step 45 that tests the status. Step 46 changes the status from a READY status to SAMPLING status during an initial iteration during the testing mode. Step 46 then transfers to step 47. During subsequent iterations step 45 transfers control directly to step 47 because the corresponding DPM unit will be in a SAMPLING mode.

Step 47 reads the data from the selected DPM unit and records the reading in the input data buffer 35 of the memory 34. Step 50 updates the display for the selected DPM unit thereby displaying the READY message for the DPM unit on a corresponding display.

Typically during a patient diagnosis readings are accumulated for a selected time interval. Step 51 determines the time that remains for such a testing interval for the selected DPM unit. If additional time remains, step 52 transfers control to step 43. If the status for all the DPM units has shifted to an “OK” state, no additional recording is needed so step 43 transfers control to step 53 and terminates the measurement.

However, if at least one DPM unit is still in a sampling state, control passes from step 43 to step 44 to select a next active channel before control transfers back to step 42. If a selected channel has completed the test, step 43 transfers directly to step 44.

When the interval for testing has been completed, step 52 transfers control to step 54 to change the status for that DPM unit to “OK” to indicate completion of the test with that DPM unit. Step 54 also updates the status display for the corresponding DPM unit.

As will now be apparent, the configuration shown in FIG. 3 taken in conjunction with the functional diagram of FIG. 4 depicts one embodiment of a system that allows concurrent relative hydration measurements from multiple DPM units. More specifically, the procedure of FIG. 4 obtains data sequentially from each of the DPM units during the operation of the iterative loop. During each operation the display 24 is updated. Consequently the display 24 provides a “real time” display for each DPM unit being monitored.

This invention has been disclosed in terms of certain embodiments. It will be apparent that many modifications can be made to the disclosed apparatus without departing from the invention. Therefore, it is the intent of the appended claims to cover all such variations and modifications as come within the true spirit and scope of this invention.

1. A method for recording in a data processing system with a plurality of serial ports, a memory and a display, relative hydration readings from a plurality of dermal phase meter units concurrently comprising:
A) establishing a concordance between each of the DPM units and one of the serial ports;
B) iteratively identifying each of the serial ports in the concordance and during each iteration,
i) selecting the corresponding DPM unit,
ii) reading the data from the selected dpm unit,
iii) displaying the reading from the dpm unit on a channel of the display, and
iv) recording the reading from the dpm unit in the memory:

2. A method as recited in claim 1 wherein each dermal phase meter units is characterized by an operating status and wherein each serial port identification includes the step of testing the operating status.

3. A method as recited in claim 2 wherein each serial port and corresponding dermal phase meter unit is characterized by a testing operating mode, said method disabling said identification of the selected corresponding dermal phase meter unit if corresponding operating mode is in other than the testing operating mode.

4. A method as recited in claim 3 additionally including updating the status for a selected serial port and corresponding dermal phase meter unit to a sampling status during the steps of taking a first reading when during the testing operating mode.

5. A method as recited in claim 4 wherein the readings from a selected serial port and corresponding dermal phase meter are to continue for a predetermined time, said method terminating the testing upon completion of the predetermined time and updating the status.

6. Apparatus for recording in a data processing system with a plurality of serial ports, a memory and a display, relative hydration readings from a plurality of dermal phase meter units concurrently comprising:
A) means for establishing a concordance between each of said units and one of the serial ports;
B) means for iteratively identifying each of the serial ports in said concordance means, and
C) processing means operation during each iteration including:
i) means for selecting the corresponding unit,
ii) means for reading the data from the selected unit,
iii) means for displaying the reading from the unit on a channel of the display, and
iv) means for recording the reading from the unit in the memory.

7. Recording apparatus as recited in claim 6 wherein each dermal phase meter unit is characterized by an operating status and wherein each serial port identification includes means for testing the operating status.

8. Recording apparatus as recited in claim 7 wherein each serial port and corresponding dermal phase meter unit is characterized by a testing operating mode, said apparatus including means for disabling said identification of the selected corresponding dermal phase meter unit if said testing means indicates that corresponding operating mode is in other than the testing operating mode.

9. Recording apparatus as recited in claim 8 additionally including means for updating the status for a selected serial port and corresponding dermal phase meter unit to a sampling status during a first reading during the testing operating mode.

10. Recording apparatus as recited in claim 9 wherein the readings from a selected serial port and corresponding dermal phase meter are to continue for a predetermined time, said apparatus including means for terminating the testing upon completion of the predetermined time and updating the status.
